(19) United States
(12) Patent Application Publication
(10) Pub. No.: US 2023/0173011 A1
(43) Pub. Date: Jun. 8, 2023

(54) APPLICATION OF TRADITIONAL CHINESE MEDICINE COMPOSITION TO PREPARATION OF MEDICINE FOR TREATING OR PREVENTING CORONAVIRUS INFECTION

(71) Applicant: JIANGSU KANION PHARMACEUTICAL CO., LTD., Lianyungang, Jiangsu (CN)

(72) Inventors: Wei XIAO, Lianyungang (CN); Zhenzhen SU, Lianyungang (CN); Xiaoqian TAO, Lianyungang (CN); Zhipeng KE, Lianyungang (CN); Xinzhuang ZHANG, Lianyungang (CN); Zeyu CAO, Lianyungang (CN); Yi DENG, Lianyungang (CN); Liang CAO, Lianyungang (CN); Zhenzhong WANG, Lianyungang (CN); Xiaolian HE, Lianyungang (CN); Xuehong DONG, Lianyungang (CN); Yanqiu WANG, Lianyungang (CN)

(73) Assignee: JIANGSU KANION PHARMACEUTICAL CO., LTD., Lianyungang, Jiangsu (CN)

(21) Appl. No.: 17/920,165

(22) PCT Filed: Mar. 11, 2021
(86) PCT No.: PCT/CN2021/080153

§ 371 Date: Oct. 20, 2022

(30) Foreign Application Priority Data
Apr. 23, 2020 (CN) 202010326218.1

(51) Int. Cl. A61K 36/534; A61K 36/355; A61K 36/634; A61K 36/538; A61K 36/28; A61K 36/346; A61K 36/899; A61K 36/484; A61P 31/14

(52) U.S. Cl. CPC A61K 36/534; A61K 36/355; A61K 36/634; A61K 36/538; A61K 36/28; A61K 36/346; A61K 36/899; A61K 36/484; A61P 31/14; A61K 2236/331; A61K 2236/333; A61K 2236/39; A61K 2236/51

(57) ABSTRACT

Application of a traditional Chinese medicine composition to preparation of a medicine for treating or preventing coronavirus infection. The traditional Chinese medicine composition includes: 2-10 parts of flos lonicerae, 2-10 parts of fructus forsythiae, 2-6 parts of herba menthae, 2-4 parts of herba schizonepetae, 2-5 parts of fermented soybean, 2-6 parts of fried arctii fructus, 2-6 parts of radix platycodonis, 2-4 parts of herba lophatheri and 2-5 parts of radix glycyrrhizae. The traditional Chinese medicine composition has a good effect of treating or preventing coronavirus-related diseases.

### TECHNICAL FIELD

The present application relates to the field of medicines for treating diseases, and in particular to application of a traditional Chinese medicine composition to preparation of a medicine for treating or preventing coronavirus infection.

### BACKGROUND

A viral pneumonia of unknown cause has the clinical manifestations: fever, cough, polypnea and dyspnea. In severe cases, it will lead to severe acute lung injury, acute respiratory distress syndrome, multiple organ failure, even death. The World Health Organization (WHO) named the virus causing the disease as “severe acute respiratory syndrome coronavirus 2 (SARS-CoV-2)”, and the viral pneumonia as COVID-19.

How to effectively treat this disease is a key problem to be solved urgently. At present, there is no effective vaccine or medicine to prevent and treat this disease. Traditional Chinese medicine has played an important role in the process of preventing and treating fulminating infectious diseases such as “epidemic diseases” since ancient times. Traditional Chinese medicine is mainly based on compound application, has the characteristics of multi-ingredient and multi-target synergistic effects, and has the integrated comprehensive regulating effect of broad-spectrum antibacterial and anti-virus properties and immune function regulation. In this epidemic situation of COVID-19, a large number of traditional Chinese medicines have been applied clinically, showing a clear curative effect.

Yinqiao Jiedu Soft Capsule related to the present application is mainly prepared from nine medicines such as flos lonicerae, fructus forsythiae, herba menthae, herba schizonepetae, sojae semen praeparatum, arctii fructus (fried), radix platycodonis, herba lophatheri and radix glycyrrhizae, and has the effects of dispelling wind and relieving exterior syndrome, and clearing away heat and toxic materials. Yinqiao Jiedu Soft Capsule is clinically used to treat influenza, acute tonsillitis, pharyngolaryngitis, pneumonia, herpes, measles, mumps and other viral diseases. Modern pharmacological experiments show that Yinqiao Jiedu Soft Capsule can obviously inhibit influenza virus and respiratory syncytial virus infection. It is urgent to carry out scientific research on whether Yinqiao Jiedu Soft Capsule can effectively prevent and treat coronavirus-related diseases, especially COVID-19.

### SUMMARY

In view of this, the present application aims to provide application of a traditional Chinese medicine composition to preparation of a medicine for treating or preventing coronavirus infection, where the traditional Chinese medicine composition includes:

2-10 parts of flos lonicerae, 2-10 parts of fructus forsythiae, 2-6 parts of herba menthae, 2-4 parts of herba schizonepetae,2-5 parts of fermented soybean, 2-6 parts of fried arctii fructus, 2-6 parts of radix platycodonis, 2-4 parts of herba lophatheri and 2-5 parts of radix glycyrrhizae;

further, the traditional Chinese medicine composition includes: 4-10 parts of flos lonicerae, 4-10 parts of fructus forsythiae, 4-6 parts of herba menthae, 3-4 parts of herba schizonepetae, 3-5 parts of fermented soybean, 3-6 parts of fried arctii fructus, 3-6 parts of radix platycodonis, 3-4 parts of herba lophatheri and 3-5 parts of radix glycyrrhizae;

further, the traditional Chinese medicine composition includes: 4-8 parts of flos lonicerae, 4-8 parts of fructus forsythiae, 2-4 parts of herba menthae, 2-4 parts of herba schizonepetae, 2-3 parts of fermented soybean, 2-4 parts of fried arctii fructus, 2-4 parts of radix platycodonis, 2-4 parts of herba lophatheri and 2-3 parts of radix glycyrrhizae;

further, the traditional Chinese medicine composition includes: 4-6 parts of flos lonicerae, 4-6 parts of fructus forsythiae, 2-3 parts of herba menthae, 2-3 parts of herba schizonepetae, 2-3 parts of fermented soybean, 2-3 parts of fried arctii fructus, 2-3 parts of radix platycodonis, 2-3 parts of herba lophatheri and 2-3 parts of radix glycyrrhizae;

further, the traditional Chinese medicine composition includes: 5 parts of flos lonicerae, 5 parts of fructus forsythiae, 3 parts of herba menthae, 2 parts of herba schizonepetae, 2.5 parts of fermented soybean, 3 parts of fried arctii fructus, 3 parts of radix platycodonis, 2 parts of herba lophatheri and 2.5 parts of radix glycyrrhizae; and

further, the diseases infected with coronavirus include COVID-19 (novel coronavirus pneumonia).

More specifically, the above composition may be prepared by the following method:

(1) adding 70%-90% ethanol into the flos lonicerae, performing reflux extraction 1-3 times, for 0.5-2 hours at each time, combining the extracting solutions, recovering the ethanol, and performing concentration to obtain a thick paste, namely, flos lonicerae thick paste;

(2) adding water into the fermented soybean and boiling, performing leaching at the temperature of 70° C-90° C. 1-3 times, for 1-3 hours at each time, combining the lixiviating solutions, and filtering to obtain a fermented soybean extracting solution;

(3) extracting volatile oil from the herba menthae, the herba schizonepetae and the fructus forsythiae to obtain volatile oil for future use, collecting an aqueous solution after distillation in another container to obtain a mixed extracting solution I; decocting the medicine residues after distillation, the fried arctii fructus, the herba lophatheri, the radix glycyrrhizae and the radix platycodonis with water 1-3 times, for 1-3 hours at each time, combining the decoction, and filtering to obtain a mixed extracting solution II; and

(4) combining the fermented soybean extracting solution, the mixed extracting solution I and the mixed extracting solution II, performing concentration to obtain an extract, performing centrifugation to obtain an extract I, continuously concentrating the supernatant to obtain an extract II, combining the extract I, the extract II and the flos lonicerae thick paste, and adding the volatile oil.

More specifically, the above composition may be prepared by the following method:

adding 80% ethanol into the flos lonicerae, performing reflux extraction twice, for 1 hour at each time, filtering, combining the filtrates, recovering the ethanol, and performing concentration to obtain thick paste with a relative density being 1.28-1.30 (80° C.); adding water into the fermented soybean and boiling, performing lixiviating at the temperature of 80° C. twice, for 2 hours at each time, filtering, and combining the filtrates for further use; extracting volatile oil from the herba menthae, the herba schizonepetae and the fructus forsythiae, and collecting an aqueous solution after distillation in another container; decocting the medicine residues, the arctii fructus, the herba lophatheri, the radix glycyrrhizae and the radix platycodonis with water twice, for 2 hours at each time, filtering, and combining the filtrates; and combining the above medicinal liquid, performing concentration to obtain a clear paste with a relative density being 1.18-1.20 (80° C.), performing centrifugation, concentrating the supernatant after centrifugation to obtain a thick paste with a relative density being 1.28-1.30 (80° C.), combining the thick paste and the flos lonicerae thick paste, drying under reduced pressure, crushing the paste into fine powder, and adding the volatile oil.

Specifically, the medicine for treating or preventing coronavirus infection includes an oral administration dosage form, an injection administration dosage form or an external administration dosage form.

Specifically, the medicine for treating or preventing coronavirus infection includes but is not limited to decoction, tablet, capsule, granule, pill, injection, condensed decoction, suspending agent, dispersing agent, syrup, suppository, gel, aerosol, patch and oral liquid.

Specifically, when selected from a soft capsule, the medicine for treating or preventing coronavirus infection may be prepared by the following method:

adding 80% ethanol into the flos lonicerae, performing reflux extraction twice, for 1 hour at each time, filtering, combining the filtrates, recovering the ethanol, and performing concentration to obtain a thick paste with a relative density being 1.28-1.30 (80° C.); adding water into the fermented soybean and boiling, performing lixiviating at the temperature of 80° C. twice, for 2 hours at each time, filtering, and combining the filtrates for further use; extracting volatile oil from the herba menthae, the herba schizonepetae and the fructus forsythiae, and collecting an aqueous solution after distillation in another container; decocting the medicine residues, the arctii fructus, the herba lophatheri, the radix glycyrrhizae and the radix platycodonis with water twice, for 2 hours at each time, filtering, and combining the filtrates; and combining the medicinal liquid (including the fermented soybean extracting filtrate, the aqueous solution, and the filtrate of the medicine residues, the arctii fructus, the herba lophatheri, the radix glycyrrhizae and the radix platycodonis), performing concentration to obtain a clear paste with a relative density being 1.18-1.20 (80° C.), performing centrifugation, concentrating the supernatant after centrifugation to obtain thick paste with a relative density being 1.28-1.30 (80° C.), combining the thick paste and the flos lonicerae thick paste, drying under reduced pressure, crushing the paste into fine powder, adding the volatile oil, a proper amount of soybean oil and a proper amount of auxiliary materials, mixing the above materials uniformly, sieving and pressing the mixture into soft capsules.

The raw material medicines used in the present application may be directly ground into powder, or may be extracts prepared by a conventional method in the field; and in the composition, several components may be directly ground into powder according to a traditional method, or extracts may be prepared by the conventional method, thereby being prepared into various dosage forms, achieving better curative effect and being more conducive to being prepared into modern medicine dosage forms.

According to the present application, the inhibition effects of the traditional Chinese medicine composition on the coronavirus SARS-CoV-2 at different concentrations are investigated on VeroE6 cells. The experimental results show that at the concentrations of 2000 μg/ml, 1000 μg/ml and 500 μg/ml, the traditional Chinese medicine composition has good protective effect on the VeroE6 cells infected with the SARS-CoV-2 virus, and can inhibit the replication of the SARS-CoV-2 in cells, so that the cytopathic rate after infection is reduced, indicating that the traditional Chinese medicine composition provided by the present application has a good effect of treating or preventing coronavirus-related diseases.

### DETAILED DESCRIPTION OF THE EMBODIMENTS

The present application provides application of a traditional Chinese medicine composition. Those skilled in the art may learn from the contents of the specification to appropriately improve process parameters. It should be noted that all the similar substitutions and modifications are obvious to those skilled in the art and should be considered to be included in the present application. The method and application of the present application have been described by preferred embodiments, and relevant personnel obviously can modify or appropriately change and combine the method and application of the specification without departing from the content, spirit and scope of the present application to implement and apply the technology of the present application.

Unless otherwise specified, medicines, biomaterials or instruments used in the present application are all common commercial products and can be purchased in the market.

The present application is further described below with reference to embodiments.

### Embodiment 1: Preparation of Traditional Chinese Medicine Compound Capsule

The compound composition is: 400 g of flos lonicerae, 400 g of fructus forsythiae, 240 g of herba menthae, 160 g of herba schizonepetae, 200 g of fermented soybean, 240 g of arctii fructus (fried), 240 g of radix platycodonis, 160 g of herba lophatheri and 200 g of radix glycyrrhizae.

80% ethanol was added into the flos lonicerae, reflux extraction was performed twice, for 1 hour at each time, filtering was performed, the filtrates were combined, the ethanol was recovered, and concentration was performed to obtain a thick paste with a relative density of 1.28-1.30 (80° C.);

water was added into the fermented soybean and boiled, lixiviating was performed at 80° C. twice, for 2 hours at each time, filtering was performed, and the filtrates were combined for future use;

volatile oil was extracted from the herba menthae, the herba schizonepetae and the fructus forsythiae, and an aqueous solution after distillation was collected in another container;

the medicine residues, the arctii fructus, the herba lophatheri, the radix glycyrrhizae and the radix platycodonis were decocted with water twice, for 2 hours at each time, filtering was performed, and the filtrates were combined; and the above medicinal liquid (including the fermented soybean extracting filtrate, the aqueous solution, and the filtrate of the medicine residues, the arctii fructus, the herba lophatheri, the radix glycyrrhizae and the radix platycodonis) was combined and concentrated to obtain a clear paste with a relative density being 1.18-1.20 (80° C.), centrifugation was performed to obtain an extract I, the supernatant after centrifugation was continuously concentrated to obtain a thick paste with a relative density being 1.28-1.30 (80° C.), namely, an extract II, the extract I, the extract II and the flos lonicerae thick paste were combined, dried under reduced pressure and crushed into fine powder, the volatile oil, a proper amount of soybean oil and a proper amount of auxiliary materials were added, mixed uniformly, sieved and pressed into 1000 soft capsules, 0.45 g/capsule.

### Embodiment 2: Pharmaceutical Effect Experimental Example

### 1. Experimental Materials

Test medicine: samples prepared in Embodiment 1.

Cell: VeroE6 cells, preserved in Viral Laboratory of State Key

Laboratory of Respiratory Disease of Guangzhou Institute of Respiratory Health.

Virus: SARS-CoV-2, the titer was TCID₅₀=10⁻⁶/100 μL, preserved in Guangzhou Customs Technical Center BSL-3 Laboratory (Laboratory of Highly Pathogenic Microorganism of State Key Laboratory of Respiratory Disease) at −80° C.; and the virus titer was 100TCID₅₀.

### 2. Experimental Method

100 μl of VeroE6 cells with the concentration of 2×10⁵ cells/mL was added into each well of a sterile 96-well culture plate, and cultivation was performed under the conditions of 37° C. and 5% CO₂ for 24 hours; 100 TCID₅₀ virus liquid (100 μL/well) was added into a culture plate experimental group and a virus control group, and virus adsorption was performed in an incubator under 37° C. and 5% CO₂ for 2 hours; after 2 hours, cell culture fluid in the 96-well culture plate was discarded; the test medicine was diluted into three concentrations such as 2000 μg preparation/ml, 1000 μg preparation/ml and 500 μg preparation/ml, three duplicate-wells were set for each concentration, and 100 μl of the above medicinal liquid was added in each well; meanwhile, cell control, blank control (solvent control), virus control (negative control) and positive medicine control group (chloroquine or remdesivir) were set;

cells were incubated in the incubator under the conditions of 37° C. and 5% CO₂ for 3-4 days; cytopathic effect (CPE) was observed under an optical microscope, and the degree of the cytopathic effect was recorded as the following six levels: “−” means that no cytopathic effect occurs, “±” means that the cytopathic effect rate is less than 10%; “+” means that the cytopathic effect rate is about 25%; “++” means that the cytopathic effect rate is about 50%; “+++” means that the cytopathic effect rate is about 75%; and “++++” means that the cytopathic effect rate is greater than 75%. All the experimental operations were completed in the BSL-3 laboratory.

### 3. Result Judgment

The inhibition rate (%) of the medicine on the virus was calculated according to the Reed-Muench method.

### 4. Experimental Result

At the three concentrations of the medicine group according to the present application, the replication of SARS-CoV-2 can be effectively inhibited in cell experiments. The results are shown in Table 1.

TABLE 1
Anti-2019-nCoV pharmaceutical effect of medicine

| Medicine Concentration | |
|---|---|
| (μg/ml) | Inhibition Rate (%) |
| 2000.00 | 66.67 ± 14.43 |
| 1000.00 | 41.67 ± 16.07 |
| 500.00 | 20.00 ± 8.66 |

### 5. Conclusion

According to the screening results of cell level, the samples prepared according to the present application can inhibit the replication of SARS-CoV-2 in the cells at the concentrations of 2000 μg/ml, 1000 μg/ml and 500 μg/ml, indicating that the medicine provided by the present application has good anti-SARS-CoV-2 activity in vitro.

It should be noted that those skilled in the art may learn from the contents of the specification to appropriately improve process parameters, and all the similar substitutions and modifications are obvious to those skilled in the art and should be considered to be included in the present application.

1-9. (canceled)

10. Application of a traditional Chinese medicine composition to preparation of a medicine for treating or preventing coronavirus infection, wherein the traditional Chinese medicine composition comprises: 2-10 parts of flos lonicerae, 2-10 parts of fructus forsythiae, 2-6 parts of herba menthae, 2-4 parts of herba schizonepetae, 2-5 parts of fermented soybean, 2-6 parts of fried arctii fructus, 2-6 parts of radix platycodonis, 2-4 parts of herba lophatheri and 2-5 parts of radix glycyrrhizae.

11. The application according to claim 10, wherein the coronavirus infection comprises COVID-19 infection.

12. The application according to claim 10, wherein the traditional Chinese medicine composition comprises: 4-10 parts of flos lonicerae, 4-10 parts of fructus forsythiae, 4-6 parts of herba menthae, 3-4 parts of herba schizonepetae, 3-5 parts of fermented soybean, 3-6 parts of fried arctii fructus, 3-6 parts of radix platycodonis, 3-4 parts of herba lophatheri and 3-5 parts of radix glycyrrhizae.

13. The application according to claim 10, wherein the traditional Chinese medicine composition comprises: 4-8 parts of flos lonicerae, 4-8 parts of fructus forsythiae, 2-4 parts of herba menthae, 2-4 parts of herba schizonepetae, 2-3 parts of fermented soybean, 2-4 parts of fried arctii fructus, 2-4 parts of radix platycodonis, 2-4 parts of herba lophatheri and 2-3 parts of radix glycyrrhizae.

14. The application according to claim 10, wherein the traditional Chinese medicine composition comprises: 5 parts of flos lonicerae, 5 parts of fructus forsythiae, 3 parts of herba menthae, 2 parts of herba schizonepetae, 2.5 parts of fermented soybean, 3 parts of fried arctii fructus, 3 parts of radix platycodonis, 2 parts of herba lophatheri and 2.5 parts of radix glycyrrhizae.

15. The application according to claim 10, wherein the medicine for treating or preventing coronavirus infection comprises an oral administration dosage form, an injection administration dosage form or an external administration dosage form.

16. The application according to claim 15, wherein the medicine for treating or preventing coronavirus infection comprises decoction, tablet, capsule, granule, pill, injection, condensed decoction, suspending agent, dispersing agent, syrup, suppository, gel, aerosol, patch and oral liquid.

17. The application according to claim 10, wherein a preparation method of the traditional Chinese medicine composition comprises the following steps:
(1) adding 70%-90% ethanol into the flos lonicerae, performing reflux extraction 1-3 times for 0.5-2 hours at each time, combining the extracting solutions, recovering the ethanol, and performing concentration to obtain a flos lonicerae thick paste;
(2) adding water into the fermented soybean and boiling, performing lixiviating at a temperature of 70° C-90° C. 1-3 times for 1-3 hours at each time, combining the lixiviating solutions, and filtering to obtain a fermented soybean extracting solution;
(3) extracting volatile oil from the herba menthae, the herba schizonepetae and the fructus forsythiae to obtain volatile oil for future use, collecting an aqueous solution after distillation in another container to obtain a mixed extracting solution I; decocting the medicine residues after distillation, the fried arctii fructus, the herba lophatheri, the radix glycyrrhizae and the radix platycodonis with water 1-3 times for 1-3 hours at each time, combining the decoction, and filtering to obtain a mixed extracting solution II; and
(4) combining the fermented soybean, the mixed extracting solution I and the mixed extracting solution II, performing concentration to obtain an extract, performing centrifugation to obtain an extract I, continuously concentrating the supernatant after centrifugation to obtain an extract II, combining the extract I, the extract II and the flos lonicerae thick paste, and adding the volatile oil.

18. The application according to claim 17, wherein the preparation method of the traditional Chinese medicine composition comprises the following steps:
adding 80% ethanol into the flos lonicerae, performing reflux extraction twice for 1 hour at each time, filtering, combining the filtrates, recovering the ethanol, and performing concentration to obtain a thick paste with a relative density being 1.28-1.30 at 80° C.; adding water into the fermented soybean and boiling, performing lixiviating at the temperature of 80° C. twice for 2 hours at each time, filtering, and combining the filtrates for further use; extracting volatile oil from the herba menthae, the herba schizonepetae and the fructus forsythiae, and collecting an aqueous solution after distillation in another container; decocting the medicine residues, the arctii fructus, the herba lophatheri, the radix glycyrrhizae and the radix platycodonis with water twice for 2 hours at each time, filtering, and combining the filtrates; and combining the medicinal liquid, performing concentration to obtain a clear paste with a relative density being 1.18-1.20 at 80° C., continuously concentrating the supernatant after centrifugation to obtain a thick paste with a relative density being 1.28-1.30 at 80° C., combining the thick paste and the flos lonicerae thick paste, drying under reduced pressure, crushing the paste into fine powder, and adding the volatile oil.
